(19) United States
(12) Patent Application Publication
(10) Pub. No.: US 2018/0110842 A1
(43) Pub. Date: Apr. 26, 2018

(54) COMPOSITIONS COMPRISING ADAMTS13 FOR USE IN METHODS FOR THE RECANALIZATION OF OCCLUDED BLOOD VESSELS IN AN INFARCTION

(71) Applicants: Baxalta Incorporated, Bannockburn, IL (US); Baxalta GmbH, Zug (CH); Katholieke Universiteit Leuven, Leuven (BE)

(72) Inventors: Barbara PLAIMAUER, Vienna (AT); Hanspeter ROTTENSTEINER, Vienna (AT); Friedrich SCHEIFLINGER, Wien (AT); Simon DE MEYER, Hooglede (BE); Frederik DENORME, Kortrijk (BE)

(73) Assignees: Baxalta Incorporated, Bannockburn, IL (US); Baxalta GmbH, Zug (CH); Katholieke Universiteit Leuven, Leuven (BE)

(21) Appl. No.: 15/572,681

(22) PCT Filed: May 26, 2016
(86) PCT No.: PCT/US16/34353

§ 371 Date: Nov. 8, 2017

Related U.S. Application Data
(60) Provisional application No. 62/166,586, filed on May 26, 2015.

(51) Int. Cl. A61K 38/48; A61P 9/10

(52) U.S. Cl. CPC A61K 38/4886; A61P 9/10; C12Y 304/24087

(57) ABSTRACT

Provided herein are methods for recanalization of occluded blood vessels in a subject having an infarction. The method includes a step of administering to the subject a therapeutically effective amount of isolated ADAMTS13 protein at particular dosages and ranges of times after detection of the infarction. As described herein, ADAMTS13 advantageously recanalizes occluded blood vessels and reduces infarction size, even when administered a prolonged period after stable occlusion. Accordingly, such methods and compositions are useful for the treatment of infractions caused by blood vessel occlusion.

### CROSS-REFERENCES TO RELATED APPLICATIONS

This application is a national phase of International Application No. PCT/US2016/034353, filed May 26, 2016, which claims priority to U.S. Provisional Application No. 62/166,586, filed May 26, 2015, the disclosures of which are incorporated herein by reference in their entirety.

### TECHNICAL FIELD

Provided herein are methods and compositions for recanalization of occluded blood vessels in a subject having an infarction. The method includes a step of administering to the subject a therapeutically effective amount of isolated ADAMTS13 protein at particular dosages and ranges of times after detection of the infarction. As described herein, ADAMTS13 advantageously recanalizes occluded blood vessels and reduces infarction size, even when administered a prolonged period after stable occlusion. Accordingly, such methods and compositions are useful for the treatment of infractions caused by blood vessel occlusion.

### BACKGROUND

An infarction is the process resulting in a macroscopic area of necrotic tissue in an organ caused by loss of adequate blood supply. Supplying arteries can be blocked from within by some obstruction (e.g., a blood clot or fatty cholesterol deposit), or can be mechanically compressed or ruptured by trauma. Infarctions are commonly associated with atherosclerosis, where an atherosclerotic plaque ruptures, a thrombus forms on the surface occluding the blood flow and occasionally forming an embolus that occludes other blood vessels downstream. Infarctions in some cases involve mechanical blockage of the blood supply, such as when part of the gut herniates or twists.

Infarctions can be generally divided into two types according to the amount of hemorrhaging present: one type is anemic infarction, which affects solid organs such as the heart, spleen, and kidneys. The occlusion is most often composed of platelets, and the organ becomes white, or pale. The second is hemorrhagic infarctions, affecting, e.g., the lungs, brain, etc. The occlusion consists more of red blood cells and fibrin strands.

Because of the serious and irreversible nature of organ damage in infarctions, there exists a clear need for new and effective methods to reduce the level and extent of an infarction.

### SUMMARY

Provided herein are methods for recanalization of occluded blood vessels in a subject having an infarction. The method includes a step of administering to the subject a therapeutically effective amount of isolated ADAMTS13 protein at particular dosages and ranges of times after detection of the infarction. As described herein, ADAMTS13 advantageously recanalizes occluded blood vessels and reduces infarction size, even when administered a prolonged period after stable occlusion. Accordingly, such methods and compositions are useful for the treatment of infarctions caused by blood vessel occlusion. In exemplary embodiments, the infarction is a cerebral infarction.

In one aspect, provided herein is a method for recanalization of an occluded blood vessel in a subject having an infarction. The method includes a step of administering to the subject a pharmaceutical composition comprising a therapeutically effective amount of isolated ADAMTS13 protein, thereby recanalizing the occluded blood vessel. In this method, the pharmaceutical composition is administered to the subject at a dose of 40, 50, 60, 70, 80, 90, 100, 150, 200, 250, 300, 350, 400, 450, 500, 550, 600, 650, 700, 750, 800, 850, 900, 950, 1,000, 1,250, 1,500, 1,750, or 2,000 U/kg. In exemplary embodiments, the infarction is a cerebral infarction.

In a second aspect, provided herein is a method for recanalization of an occluded blood vessel in a subject having an infarction. This method includes a step of administering to the subject a pharmaceutical composition comprising a therapeutically effective amount of isolated ADAMTS13 protein, thereby recanalizing the occluded blood vessel. In this method, the pharmaceutical composition is administered to the subject within 15, 30, 60, 90, 120, 180, 210, 240, 270 or 300 minutes of detection of the infarction. In exemplary embodiments, the infarction is a cerebral infarction.

In a third aspect, provided herein is a method for treating an infarction in a subject by recanalization of an occluded blood vessel in the subject. The method includes a step of administering to the subject a pharmaceutical composition that includes a therapeutically effective amount of isolated ADAMTS13 protein, thereby treating the infarction. In such a method, the pharmaceutical composition is administered to the subject at a dose of about 40, 50, 60, 70, 80, 90, 100, 150, 200, 250, 300, 350, 400, 450, 500, 550, 600, 650, 700, 750, 800, 850, 900, 950, 1,000, 1,250, 1,500, 1,750, or 2,000 U/kg. In exemplary embodiments, the infarction is a cerebral infarction.

In a fourth aspect, provided herein is a method for treating an infarction in a subject by recanalization of an occluded blood vessel in the subject. The method includes a step of administering to the subject a pharmaceutical composition that includes a therapeutically effective amount of isolated ADAMTS13 protein, thereby treating the infarction. In this method, the pharmaceutical composition is administered to the subject within 15, 30, 60, 90, 120, 180, 210, 240, 270 or 300 minutes of detection of the infarction. In exemplary embodiments, the infarction is a cerebral infarction.

In some embodiments of the above subject methods, the pharmaceutical composition is administered to the subject at a dose of about 40, 50, 60, 70, 80, 90, 100, 150, 200, 250, 300, 350, 400, 450, 500, 550, 600, 650, 700, 750, 800, 850, 900, 950, 1,000, 1,250, 1,500, 1,750, or 2,000 U/kg and within 15, 30, 60, 90, 120, 180, 210, 240, 270 or 300 minutes of detection of the infarction.

In a fifth aspect, provided herein is a method for recanalization of an occluded blood vessel in a subject having an infarction. The method includes a step of administering to the subject a pharmaceutical composition that includes a therapeutically effective amount of isolated ADAMTS13 protein, thereby recanalizing the occluded blood vessel. In this method, the pharmaceutical composition is administered to the subject at an amount that increases the level of the ADAMTS13 protein in the subject 1.1, 1.2, 1.3, 1.4, 1.5, 1.6, 1.7, 1.8, 1.9, 2, 3, 4, 5, 6, 7, 8, 9, 10, 15, or 20-fold greater than the level of ADAMTS13 protein in the subject prior to the administering. In some embodiments of this method, the pharmaceutical composition administered to the subject within 15, 30, 60, 90, 120, 180, 210, 240, 270 or 300 minutes of detection of the infarction. In exemplary embodiments, the infarction is a cerebral infarction.

In certain embodiments of the subject methods, the regional cerebral blood flow in the subject is improved by at least 25% as compared to a control subject not administered the composition comprising the therapeutically effective amount of isolated ADAMTS13 protein. In some embodiments of the methods provided herein, the regional cerebral blood flow is improved by at least 50% as compared to the regional cerebral blood flow in the control. In some embodiments of the methods provided herein, the regional cerebral blood flow is improved by at least 75% as compared to the regional cerebral blood flow in the control subject.

In exemplary embodiments, the isolated ADAMTS13 protein is glycosylated. In certain embodiments, the isolated ADAMTS13 protein has a plasma half-life of more than 1 hour. In some embodiments, the isolated ADAMTS13 protein is recombinantly produced by HEK293 cells. In certain embodiments, the isolated ADAMTS13 protein is recombinantly produced by CHO cells.

In exemplary embodiments of the methods provided herein, the pharmaceutical composition is administered multiple times or by continuous infusion. In some embodiments, the administration does not increase the level of hemorrhage, as compared to the level of hemorrhage in a subject not receiving the pharmaceutical composition. In certain embodiments, the administration reduces infarct volume.

In certain embodiments, the infract volume is reduced by at least 50% compared to the infract volume in a control subject not administered the composition comprising the therapeutically effective amount of isolated ADAMTS13 protein.

In a sixth aspect, provided herein is a method of improving the recovery of sensorimotor function in a subject that has experienced a cerebral infarction. This method includes the step of administering to the subject a pharmaceutical composition that includes a therapeutically effective amount of isolated ADAMTS13 protein, where the regional cerebral blood flow in the subject is improved by at least 25% as compared to the regional cerebral blood flow in a control subject.

### BRIEF DESCRIPTION OF THE DRAWINGS

FIG. 1A-1C. FeCl₃-induced thrombotic occlusion of the right MCA. (FIG. 1A) 25× magnification of the exposed right temporal bone. Via a small local craniotomy, the right MCA is exposed and the trace of the MCA is followed across the bregma to allow blood flow monitoring using a laser Doppler flow (LDF) probe. (FIG. 1B) Thrombotic occlusion of the MCA is induced by topical application of small filter paper saturated with 20% FeCl₃ for 4 min on the MCA. (FIG. 1C) Application of FeCl₃ results in a rapid decrease of rCBF, below 25% of baseline. (FIG. 1D) Depending on the type of injury, a small (threshold injury) or a large (strong injury) white platelet rich clot is formed.

FIG. 2A-2F. ADAMTS13 is a determinant of thrombus stability in the MCA. A FeCl₃-induced injury was induced in the MCA of both ADAMTS13 KO and WT animals to cause thrombotic occlusion of the MCA. (FIG. 2A) Absence of ADAMTS13 results in a faster occlusion of the MCA. Time to first occlusion was defined as the time after FeCl₃ application until rCBF dropped below 25%. (FIG. 2B) Spontaneous dissolution of the occluding thrombus was impaired in the absence of ADAMTS13: time to first recanalization after occlusion was significantly smaller in WT mice compared to ADAMTS13 KOM mice. (FIGS. 2C-2F) Representative laser doppler flow charts of rCBF of the MCA territory show distinct differences in recanalization profiles between ADAMTS13 KO and WT mice. In FIG. 2C and FIG. 2D two representative rCBF plots of WT mice are depicted. FIG. 2C represents one in which blood flow was quickly restored to baseline values, while FIG. 2D shows a typical WT mice that is in the process of gradually restoring rCBF. In contrast, representative ADAMTS13 KO mice depicted in FIG. 2E and FIG. 2F show in FIG. 2E a permanently occluded mouse and in FIG. 2F one that is recanalizing but is unsuccessful in completely restoring rCBF to baseline values. (data represent results from 13-14 mice/group; *, p<0.05; **, p<0.01; ***, p<0.005).

FIG. 3A-3C. Administration of rhADAMTS13 enhances MCA recanalization and saves the brain from ischemic injury in ADAMTS13 KO mice. An occlusive thrombus was formed in the MCA of WT and ADAMTS13 KO mice via topical application of a threshold amount of FeCl₃, leading to thrombotic occlusion of the MCA. To a subset of ADAMTS13 KO mice, rhADAMTS13 (3500 U/kg) was administered 5 minutes after occlusion. After occlusion, rCBF was monitored via laser doppler flowmetry. Twenty-four hours after occlusion, cerebral infarctions were determined via TTC staining. (FIG. 3A) Averaged post-occlusion MCA blood flow profiles reveal that restoration of rCBF was significantly impaired in ADAMTS13 KO mice compared to WT animals. Administration of rhADAMTS13 (arrow) restored rCBF to WT values. (FIG. 3B) Representative TTC stained brain slices of ADAMTS13 KO mice, WT mice and ADAMTS13 KO mice injected with rhADAMTS13. (FIG. 3C) Scatter dot plot of infarct sizes 24 hours after occlusion. Infarct sizes of ADAMTS13 KO mice were significantly larger than those observed in WT mice. Treatment of ADAMTS13 KO mice with rhADAMTS13 5 minutes after occlusion significantly reduced infarct sizes. (n=10-14 mice/group; *, p<0.05; **, p<0.01)

FIG. 4A-4D. rhADAMTS13 exerts a protective effect on ischemic brain injury after permeant thrombotic MCA occlusion by restoring MCA blood flow of WT mice. By generating a severe FeCl₃-induced injury to the right MCA of WT C57/B16J mice, an occluding and stable thrombus was resistant to spontaneous dissolution. Five minutes after occlusion, different doses of rhADAMTS13 were intravenously administered and rCBF was monitored for 60 min. (FIG. 4A) After thrombotic occlusion of the MCA, rhADAMTS13 restores MCA rCBF in a dose dependent way. (FIG. 4B) The proportion of animals that restore rCBF levels to more than 25%, 50% or 75% increases with rhADAMTS13 dose. (FIG. 4C) When ischemic brain injury was assessed 24 h after occlusion, a dose-dependent reduction of infarct size was observed with increasing amounts of rhADAMTS13. (FIG. 4D) Representative TTC stainings of three consecutive coronal brain sections taken from mice treated with vehicle or the highest dose of rhADAMTS13 (3500 U/kg). (n=9 and 8 mice respectively for vehicle and 3500 U/kg rhADAMTS13, n=5 for the lower doses of rhADAMTS13; *, p<0.05; ***, p<0.005).

FIG. 5A-5C. Delayed rhADAMTS13 administration 60 minutes after occlusion is able to restore MCA blood and reduce ischemic brain injury. Sixty minutes after inducing stable MCA occlusion, mice were treated with either rhAMDATS13 (3500 U/kg) or vehicle (arrow). rCBF of the MCA territory was monitored via laser doppler flowmetry to assess recanalization of the MCA. (FIG. 5A) In mice treated with rhADAMTS13, a significant increase in rCBF was observed. To assess the effect on stroke outcome, infarct sizes were measured on TTC-stained brain sections. In parallel with restoration of blood flow, cerebral infarct sizes significantly decreased in mice that received rhADAMTS13. (n=8 mice in each group; *, p<0.05; ***, p<0.005)

### DEFINITIONS

The term “recanalization” refers to the restoration of the lumen of a blood vessel following an occlusion by restoration of lumen or by the formation of one or more new channels. The term “recanalizing” means restoring of the lumen of a blood vessel following an occlusion by restoration of lumen or by the formation of one or more new channels. In certain embodiments described herein, recanalization is related to an occluded blood vessel associated with an infarction (e.g., a cerebral infarction). Recanalization can be determined using any suitable method known in the art. In some embodiments where the recanalization is of an occluded cerebral blood vessel, recanalization is determined by the restoration of regional cerebral blood flow (rCBF).

“Regional cerebral blood flow” and “rCBF” refer to the amount of blood flow to a specific region of the brain in a given time. Regional cerebral blood flow can be measured using any suitable technique known in the art including, for example, using laser Doppler flow monitoring techniques described herein.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It can include regions preceding and following the coding region (leader and trailer) as well as intervening sequences (introns) between subject coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that subject substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins, W. H. Freeman and Co., N. Y. (1984)).

In the present application, amino acid residues are numbered according to their relative positions from the left most residue, which is numbered 1, in an unmodified wild-type polypeptide sequence.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used in herein, the terms “identical” or percent “identity,” in the context of describing two or more polynucleotide or amino acid sequences, refer to two or more sequences or subsequences that are the same or have a specified percentage of amino acid residues or nucleotides that are the same (for example, a core amino acid sequence responsible for NRG-integrin binding has at least 80% identity, preferably 85%, 90%, 91%, 92%, 93, 94%, 95%, 96%, 97%, 98%, 99%, or 100% identity, to a reference sequence, e.g., SEQ ID NO:1), when compared and aligned for maximum correspondence over a comparison window, or designated region as measured using one of the following sequence comparison algorithms or by manual alignment and visual inspection. Such sequences are then said to be “substantially identical.” With regard to polynucleotide sequences, this definition also refers to the complement of a test sequence. Preferably, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

An “antibody” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term “effective amount,” as used herein, refers to an amount that produces therapeutic effects for which a substance is administered. The effects include the prevention, correction, or inhibition of progression of the symptoms of a disease/condition (such as infarction) and related complications to any detectable extent. The exact amount will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

As used herein, the terms “treat” and “prevent” are not intended to be absolute terms. Treatment can refer to any delay in onset, amelioration of symptoms, improvement in patient survival, reduction of infarct volume, reduction in frequency or severity, etc. Thus, the term “treatment” can include prevention. The effect of treatment can be compared to a control, e.g., a subject or pool of subjects not receiving the treatment, an untreated tissue in the same patient, or the same subject prior to treatment.

A “biological sample” can be obtained from a patient, e.g., a biopsy, from an animal, such as an animal model, or from cultured cells, e.g., a cell line or cells removed from a patient and grown in culture for observation. Biological samples include tissue such as colorectal tissue or bodily fluids, e.g., blood, blood fractions, lymph, saliva, urine, feces, etc.

### DETAILED DESCRIPTION

### I. Use of ADAMTS13 for Recanalization of Occluded Blood Vessels

Provided herein are methods for recanalization of occluded blood vessels in a subject having an infarction (e.g. a cerebral infarction). As described herein, the present inventors have discovered that ADAMTS13 (A Disintegrin-like And Metalloprotease with Thrombospondin type I motif No. 13), is capable of restoration of blood flow (i.e. recanalization) and reduced infarction sizes in subjects having an infarction, a process in which tissue undergoes necrosis due to insufficient blood supply. ADAMTS13 advantageously exerts its effect in a dose dependent manner and these effects are observed even at prolonged periods after blood vessel occlusion.

The subject method includes a step of administering to the subject a therapeutically effective amount of an isolated ADAMTS13 protein at particular dosages and ranges of times after detection of the infarction.

The subject methods are suitable for the treatment of any infarction caused by a blood vessel occlusion. Such infarctions include, but are not limited to, a myocardial infarction, a cerebral infarction, a pulmonary infarction, a splenic infarction, a limb infarction, a bone infarction, a testicle infarction and an eye infarction.

In exemplary embodiments, the subject methods are for the recanalization of an occluded blood vessel in a subject having a cerebral infarction. “Cerebral infarction” refers to a type of ischemic stroke resulting from a blockage in the blood vessels supplying blood to the brain, which results in the death of brain tissue. Symptoms of cerebral infarction are determined by the parts of the brain affected. For example, infarcts in the primary motor cortex can cause contralateral hemiparesis. Brainstem infarcts cause brainstem syndromes including Wallenberg's syndrome, Weber's syndrome, Millard-Bubler syndrome, and Benedikt syndrome.

Recanalization of occluded blood vessels can be measured using any suitable technique. For example, recanalization can be measure by as a percentage of blood flow compared to a control baseline value (e.g., the blood flow of a control individual not having the occluded blood vessel or infarction). Blood flow can be measure, for example, using videocapillary microscoping with frame-to-frame analysis or laser Doppler anemometry techniques. See, e.g., Stucker et al. Microvascular Research 52(2): 188-192 (1996), which is incorporated herein by reference. In some embodiments, the subject method increases the blood flow by at least 10%, 15%, 20%, 25%, 30%, 35%, 40%, 45%, 50%, 55%, 60%, 65%, 70%, 75%, 80%, 85%, 90%, 95%, or 99% as compared to a control baseline value (e.g., the blood flow of a control subject not having the occluded blood vessel or infarction).

Without being bound by any particular theory of operation, it is believed that recanalization of occluded blood vessels via ADAMTS13 reduces infarct volume. In some embodiments, administration of ADAMTS13 reduces the infarct volume in the subject by at least 5% 10%, 15%, 20%, 25%, 30%, 35%, 40%, 45%, 50%, 55%, 60%, 65%, 70%, 75%, 80%, 85%, 90%, 95%, or 99% of the infarct volume of a control subject that was not administered ADAMTS13.

Features of the subject methods are described in further detail below.

### A. ADAMTS13

The subject methods provided herein include a step of administering to an individual having an infarction (e.g., a cerebral infarction) a pharmaceutical composition that includes a therapeutically effective amount of an isolated ADAMTS13 protein. ADAMTS13 (A Disintegrin-like And Metalloprotease with Thrombospondin type I motif No. 13), a 190 kDa glycosylated protein produced predominantly by the liver. ADAMTS13 is a plasma metalloprotease that cleaves VWF between tyrosine at position 1605 and methionine at position 1606, breaking down the VWF multimers into smaller units, which are further degraded by other peptidases.

As used herein, “ADAMTS13” includes biologically active derivatives of ADAMTS13. The term “biologically active derivative” as used herein means any polypeptides with substantially the same biological function as ADAMTS13, particularly in its ability. The polypeptide sequences of the biologically active derivatives can comprise deletions, additions and/or substitution of one or more amino acids whose absence, presence and/or substitution, respectively, do not have any substantial negative impact on the biological activity of polypeptide. The biological activity of said polypeptides can be measured, for example, by the reduction or delay of platelet adhesion to the endothelium or subendothelium, the reduction or delay of platelet aggregation in a flow chamber, the reduction or delay of the formation of platelet strings, the reduction or delay of thrombus formation, the reduction or delay of thrombus growth, the reduction or delay of vessel occlusion, the proteolytical cleavage of VWF, and/or the reduction of infarct volume in an experimental system similar to those described in the Examples Section of this application.

The terms “ADAMTS13” and “biologically active derivative”, respectively, also include naturally occurring polypeptides and polypeptides obtained via recombinant DNA technology. Recombinant ADAMTS13 (“rADAMTS13”), e.g., recombinant human ADAMTS13 (“r-hu-ADAMTS13”), can be produced by any method known in the art. One specific example is disclosed in WO 02/42441 with respect to the method of producing recombinant ADAMTS13. This can include any method known in the art for (i) the production of recombinant DNA by genetic engineering, e.g., via reverse transcription of RNA and/or amplification of DNA, (ii) introducing recombinant DNA into prokaryotic or eukaryotic cells by transfection, i.e., via electroporation or microinjection, (iii) cultivating said transformed cells, e.g., in a continuous or batchwise manner, (iv) expressing ADAMTS13, e.g., constitutively or upon induction, and (v) isolating said ADAMTS13, e.g., from the culture medium or by harvesting the transformed cells, in order to (vi) obtain substantially purified recombinant ADAMTS13, e.g., via anion exchange chromatography or affinity chromatography. The term “biologically active derivative” includes also chimeric molecules such as ADAMTS13 (or a biologically active derivative thereof) in combination with an immunoglobulin molecule (Ig), in order to improve the biological/pharmacological properties such as half-life of ADAMTS13 in the circulation system of a mammal, particularly human. The Ig could have also the site of binding to an Fc receptor optionally mutated.

The rADAMTS13 can be produced by expression in a suitable prokaryotic or eukaryotic host system characterized by producing a pharmacologically effective ADAMTS13 molecule. Examples of eukaryotic cells are mammalian cells, such as CHO, COS, HEK 293, BHK, SK-Hep, and HepG2. There is no particular limitation to the reagents or conditions used for producing or isolating ADAMTS13 according to the present invention and any system known in the art or commercially available can be employed. In one embodiment of the present invention, rADAMTS13 is obtained by methods as described in the state of the art. In some embodiments, the ADAMTS13 is human ADAMTS13. In certain embodiments, the ADAMTS13 is porcine ADAMTS13.

A wide variety of vectors can be used for the preparation of the rADAMTS13 and can be selected from eukaryotic and prokaryotic expression vectors. Examples of vectors for prokaryotic expression include plasmids such as pRSET, pET, pBAD, etc., wherein the promoters used in prokaryotic expression vectors include lac, trc, trp, recA, araBAD, etc. Examples of vectors for eukaryotic expression include: (i) for expression in yeast, vectors such as pAO, pPIC, pYES, pMET, using promoters such as AOX1, GAP, GAL1, AUG1, etc; (ii) for expression in insect cells, vectors such as pMT, pAc5, pIB, pMIB, pBAC, etc., using promoters such as PH, p10, MT, Ac5, OpIE2, gp64, polh, etc., and (iii) for expression in mammalian cells, vectors such as pSVL, pCMV, pRc/RSV, pcDNA3, pBPV, etc., and vectors derived from viral systems such as vaccinia virus, adeno-associated viruses, herpes viruses, retroviruses, etc., using promoters such as CMV, SV40, EF-1, UbC, RSV, ADV, BPV, and β-actin.

### B. Pharmaceutical Compositions

Also provided herein are pharmaceutical compositions useful for recanalization of blood vessels in a subject having an infarction. Such compositions comprise an effective amount of ADAMTS13 or its biologically active derivatives.

The pharmaceutical composition can comprise one or more pharmaceutically acceptable carrier and/or diluent. The pharmaceutical composition can also comprise one or more additional active ingredients such as agents that stimulate ADAMTS13 production or secretion by the treated patient/subject, agents that inhibit the degradation of ADAMTS13 and thus prolong its half-life (or alternatively glycosylated variants of ADAMTS13), agents that enhance ADAMTS13 activity (for example by binding to ADAMTS13, thereby inducing an activating conformational change), or agents that inhibit ADAMTS13 clearance from circulation, thereby increasing its plasma concentration.

It must be kept in mind that the compositions of the present invention can be employed in serious disease states, that is, life-threatening or potentially life threatening situations. In such cases, in view of the lack of side effects (e.g., hemorrhage, immune system effects), it is possible and may be felt desirable by the treating physician to administer substantial excesses of the pharmaceutical compositions of the invention.

In some embodiments, ADAMTS13 or its biologically active derivative are administered with one or more additional active ingredients such as agents that stimulate ADAMTS13 production or secretion by the treated patient/subject, agents that inhibit the degradation of ADAMTS13 and thus prolong its half-life, agents that enhance ADAMTS13 activity (for example, by binding to ADAMTS13, thereby inducing an activating conformational change), or agents that inhibit ADAMTS13 clearance from circulation, thereby increasing its plasma concentration. Another ingredient that can be co-administered include blood thinners (e.g., aspirin), anti-platelet agents, and tissue plasminogen activator (tPA), a thrombolytic serine protease that activates plasmin to cleave fibrin.

### C. Dosage Amounts and Time of Administration

The pharmaceutical compositions that are administered to the subject having an infarction contain an effective amount of ADAMTS13 protein to recanalize an occluded blood vessel. Effective amounts for the recanalization of an occluded blood vessel having an infarction (e.g., a cerebral infarction) range, for example, from 0.1 to 20 mg/kg body weight. In some embodiments, the pharmaceutical composition is administered to the subject at a dose of about 40, 50, 60, 70, 80, 90, 100, 150, 200, 250, 300, 350, 400, 450, 500, 550, 600, 650, 700, 750, 800, 850, 900, 950, 1,000, 1,250, 1,500, 1,600, 1,750, 2,000, 3000, 3500, 5000, 6000, 7000, 8000, or 10,000 U/kg body weight.

In certain embodiments, the amount of ADAMTS13 protein that is administered to the subject is measured as an increase in the amount of ADAMTS13 protein in the subject as compared to a control (e.g., the amount of ADAMTS13 protein in the subject prior to administration). In some embodiments, the ADAMTS13 protein is administered to the subject at an amount that increases the level of the ADAMTS13 protein in the subject 1.1, 1.2, 1.3, 1.4, 1.5, 1.6, 1.7, 1.8, 1.9, 2, 3, 4, 5, 6, 7, 8, 9, 10, 15, or 20-fold greater than the level of ADAMTS13 protein in the subject prior to the administering. In some embodiments, the ADAMTS13 protein is administered to the subject at an amount that increases the level of the ADAMTS13 protein at least 5%, 10%, 15%, 20%, 25%, 30%, 35%, 40%, 45%, 50%, 55%, 60%, 65%, 70%, 75%, 80%, 85%, 90%, 95% or 99% greater than the level of ADAMTS13 protein in the subject prior to the administering.

Dose can also be determined based on whether the ADAMTS13 is administered prophylactically (e.g., in repeated doses) or in response to a medical emergency, to immediately reduce harmful effects of an infarction.

The route of administration does not exhibit a specific limitation and can be, for example, subcutaneous, intraarterial, or intravenous. Oral administration of ADAMTS13 is also a possibility.

The ADAMTS13 protein can be administered to mammals, particularly humans, for prophylactic and/or therapeutic purposes. In some embodiments, the present invention is used to reduce the harmful effects of blood vessel occlusion, without increasing the likelihood of hemorrhage or disabling the peripheral immune system. In some embodiments, ADAMTS13 is administered prophylactically, e.g., to an subject at risk of a blood vessel occlusion. In such cases, prophylactic treatment is usually repeated at a lower dose for an extended period of time, e.g., for a given period of time after an initial infarction event.

Examples of subjects that can be treated according to the subject include those that have experienced an infarction, such as a heart attack, a pulmonary infarction, or stroke (e.g., a cerebral infarction), no matter the severity. This is especially true if the ADAMTS13 protein can be administered soon after the infarction, to reduce the tissue damage that results from loss of blood to the surrounding tissues. ADAMTS13 protein can be administered to subjects at a risk of experiencing infarction, e.g., as a result of illness or blood pressure related condition, surgery, or other medication.

Therapeutic administration of ADAMTS13 protein can begin at the first sign of infarction or shortly after diagnosis, e.g., to prevent recurrence. This can be followed by boosting doses for a period thereafter. In chronically affected subjects, long term treatment can be provided. In some embodiments, the pharmaceutical composition administered to the subject within 15, 30, 60, 90, 120, 180, 210, 240, 270 or 300 minutes of detection of the infarction. Symptoms with respect to cerebral infarctions are determined by the region of tissue damage. If the infarct is located in primary motor cortex, contralateral hemiparesis is said to occur. With brainstem localization, brainstem syndromes such as Wallenberg's syndrome, Weber's syndrome, Millard-Gubler syndrome, Benedikt syndrome or others are typical. Infarctions will result in weakness and loss of sensation on the opposite side of the body. Physical examination of the head area will reveal abnormal pupil dilation, light reaction and lack of eye movement on the opposite side. If the infarction occurs on the left side brain, speech will be slurred. Reflexes may be aggravated as well. As described in the examples provided herein, ADAMTS13 protein is capable of recanalization and reduction of infarction volume even at prolonged periods after blood vessel occlusion. In certain embodiments, recanalization leads to a decrease of at least 10%, 20%, 30%, 40%, or 50% in infarct volume, when compared to a control (e.g., a subject not administered ADAMTS13).

The present compositions and methods will be further illustrated in the following examples, without any limitation thereto.

### EXAMPLES

### A. Materials and Methods

### Mice

All animal studies were performed in accordance with the local ethical law and the local ethical committees (P081-2014 K U Leuven, Leuven, Belgium; act no. 87-848) and guidelines for the care and use of laboratory animals. Experiments were performed on 8 to 12 weeks old male and female ADAMTS13 KO and WT mice on a mixed C57BL/6J and 129X1/SvJ background⁷⁷ and 8 to 12 weeks old male and female C57BL/6J mice (The Jackson laboratory).

### Thrombotic Occlusion of the MCA

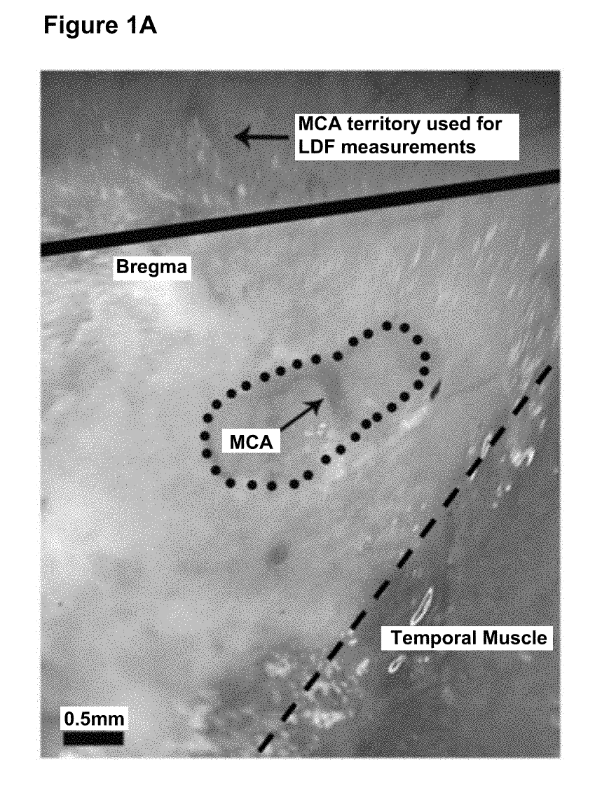

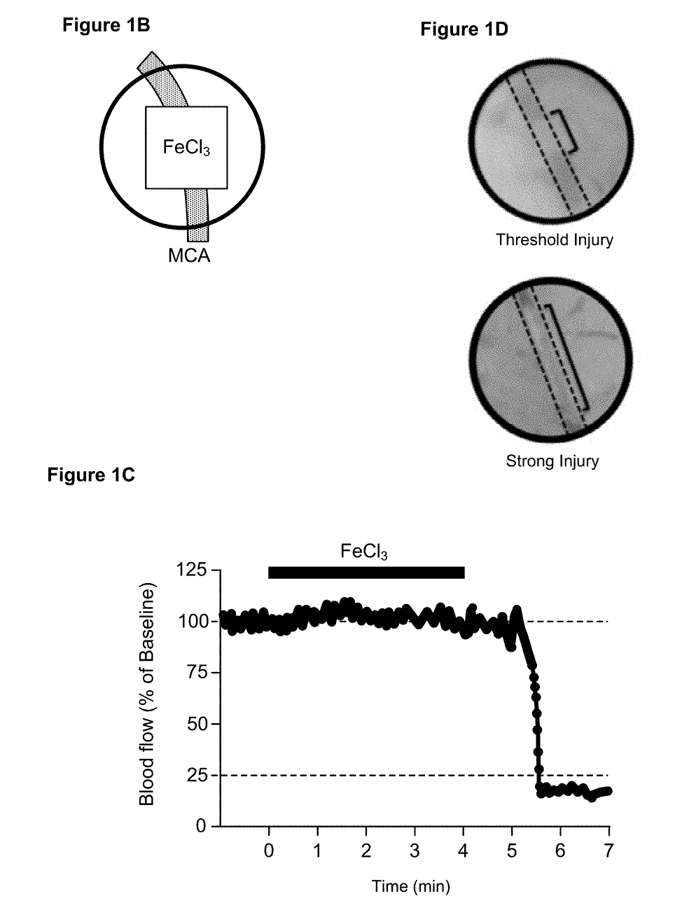

Mice were deeply anesthetized with 5% isoflurane in pure 02 and placed in a stereotaxic frame after which anesthesia was maintained with 2% isoflurane for surgical procedures and monitoring of regional cerebral blood flow (rCBF). During anesthesia, mouse body temperature was maintained at 37° C. via a rectal probe and a thermostat-controlled heating pad under the mouse (TC-1000 Temperature controller, CWE Inc., Ardmore, USA). Stroke was induced via the formation of an occlusive thrombus in the MCA as previously described with slight modifications (see Karatas et al., Journal of Cerebral Blood Flow and Metabolism 31: 1452-1460 (2011)). Via a skin incision between the right eye and ear, the temporalis muscle was excised, and a small craniotomy was performed on the parietal bone to expose the right MCA. A small piece of Whatman filter paper (GE Healthcare, Buckinghamshire, UK) saturated with 20% FeCl₃ (Sigma-Aldrich, St. Louis, USA) was placed on top of the unharmed dura mater above the MCA (FIG. 1). For threshold MCA injury, a filterpaper of 0.5×0.5 mm was used. For strong injury, the filter paper dimensions were 0.5×1.5 mm. After 4 minutes, the filter paper was removed and the MCA at the site of application was rinsed with saline to remove residual FeCl₃.

Regional cerebral blood flow (rCBF) in the MCA territory was determined by laser Doppler flow monitoring (moorVMS-LDF1; Moor Instruments; Devon, UK). Changes in rCBF were recorded using a PowerLab 8/35 data acquisition unit (ADInstruments; Oxford, UK) and calculated using LabChart software (v8.0.5; ADInstruments; Oxford, UK). rCBF was continuously measured for 10 minutes before induction of MCA occlusion to set baseline rCBF (100%). Depending on the experiment, rCBF was monitored after thrombotic occlusion of the MCA up to a maximum of 2 hours after occlusion. Occlusion time was defined as the time between initial FeCl₃ application and the moment at which rCBF drops below 25% of baseline. Recanalization was defined as a return of averaged (over 60 seconds) rCBF above 25% of baseline values.

### Measurement of Infarct Volume

Cerebral infarct volumes were determined as described (De Meyer et al., Arteriosclerosis, Thrombosis, and Vascular Biology 30, 1949-1951 (2010)). Mice were euthanized 24 hours after occlusion of the MCA. Brains were quickly removed and cut into 2-mm-thick coronal sections using a mouse brain slice matrix. The slices were stained with 2% 2,3,5-triphenyl-tetrazolium chloride (Sigma-Aldrich) in PBS to visualize healthy tissue and unstained infarctions. Sections were photographed and infarct areas (white) were analyzed via planimetry using Image J software (National Institutes of Health, Bethesda, Md.; http://imagej.nih.gov/ij/) by an experimenter who is blinded for treatment conditions.

Staining of Thrombi from Acute Ischemic Stroke Patients

Thrombi were fixed with 4% formalin overnight, embedded in paraffin and hereafter 5 μm thick slices were cut. Consecutive slices of each thrombus were rehydrated and stained with either hematoxylin and eosin (H&E; Sigma-Aldrich (St. Louis; MO; USA)), Martius Scarlet blue (MSB) or anti-VWF (rabbit anti-human VWF (Dako A0082), counterstained with hematoxylin).

### Statistical Analysis

All data are presented as mean plus or minus standard error of the mean. Statistical analysis was performed with GraphPad Prism (Version 6.Oc). An unpaired Student's T-test was used to analyze time to first occlusion/recanalization. A Student's T-test or one-way ANOVA with Bonferroni's multiple comparison test was used for statistical comparison of infarct lesions and to compare rCBF when applicable.

### B. Example 1: Absence of ADAMTS13 Promotes Occlusive Thrombus Formation and Impairs Spontaneous Recanalization

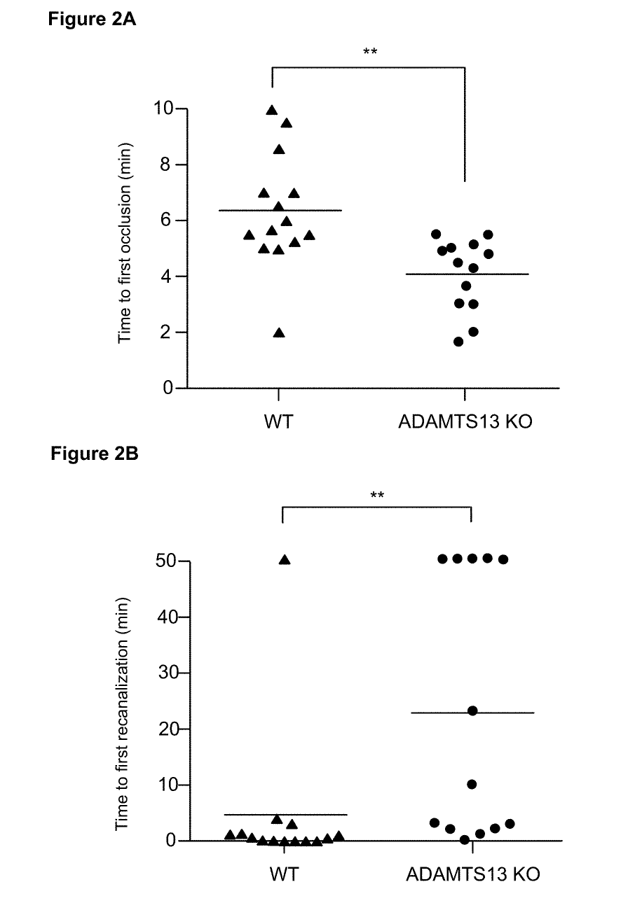

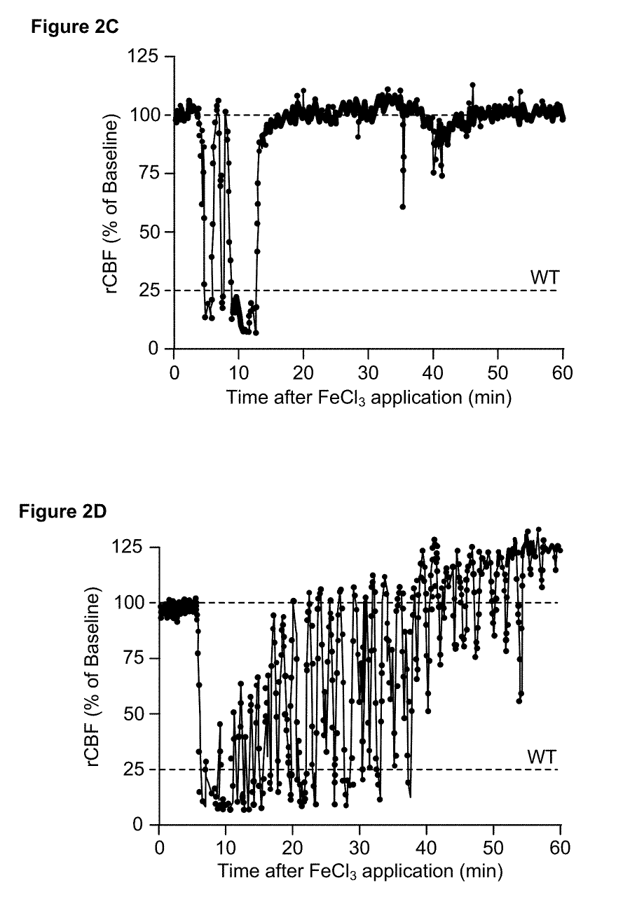

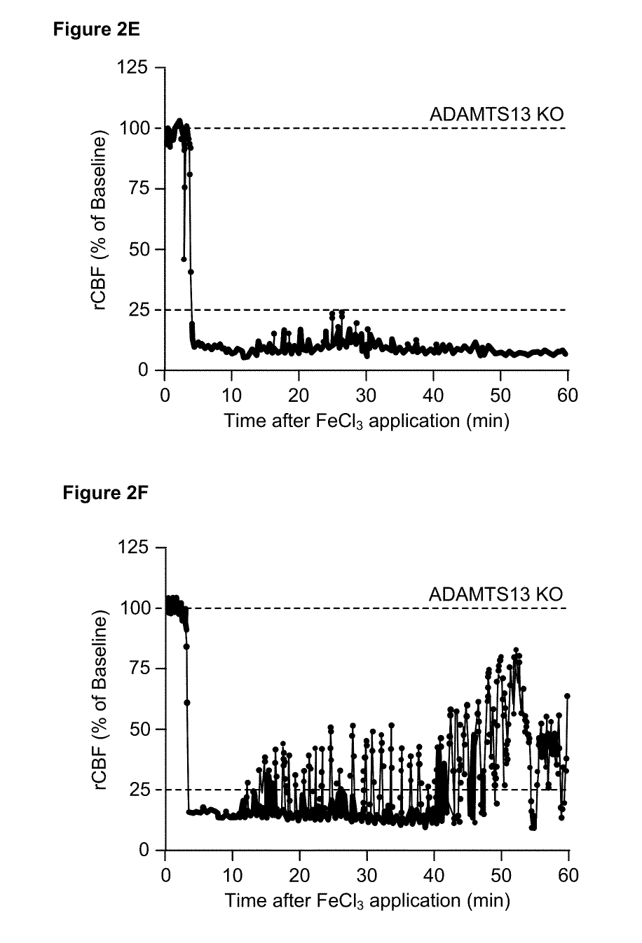

To study the effect of ADAMTS13 in thrombus dissolution, thrombotic stroke was induced in both ADAMTS13 KO mice and their wild-type (WT) littermates. In a first set of experiments, a relatively small injury to the MCA was created (using a 0.5×0.5 mm² filter paper saturated with 20% FeCl₃). Upon injury, all WT mice developed an occlusive thrombus in the MCA within 10 minutes after application of FeCl₃ (FIG. 2A). Interestingly, ADAMTS13 KO mice also developed an occlusive thrombus in the MCA, but time to occlusion was significantly shorter when compared to WT animals (4.2 min±0.5 min versus 6.4 min±0.5 min respectively, p<0.005; FIG. 2A). These data show that ADAMTS13 can delay MCA thrombus formation, probably by destabilizing the growing thrombus via cleavage of (UL-)VWF at the site of injury. Once formed, the occlusive thrombus reduced MCA blood flow to the same extent in ADAMTS13 KO and WT mice (residual blood flow of 13.4±1.4% versus 12.9±1.9% of baseline respectively, p=0.86).

Interestingly, not only did the MCA occlude faster in ADAMTS13 KO mice, also spontaneous dissolution of the occluding thrombus was significantly impaired in ADAMTS13 KO animals after threshold injury. FIG. 2B shows the time to first recanalization, defined as the time needed for restoration of rCBF above 25% of baseline. In this model, the majority of WT mice showed fast spontaneous recanalization after occlusion, with restoration of blood flow above 25% of baseline values within the first minute after occlusion. In contrast, spontaneous recanalization occurred significantly later, or did not take place at all within the experimental time frame of 50 minutes in ADAMTS13 KO mice. Whereas 79% of WT mice (11 out of 14) showed spontaneous recanalization in less than 1 minute, only 15% of ADAMTS13 KO mice showed a similarly fast recanalization (2 out of 13 animals). In addition, a distinct difference in the pattern of recanalization was observed between WT and ADAMTS13 KO mice: Whereas stable blood flow was re-established in most of the WT type mice after initial recanalization (FIG. 2C-D), recanalization in ADAMTS13 KO mice was often followed by novel thrombus formation and re-occlusion (FIG. 2E-F).

Taken together, these results show that ADAMTS13 is a determinant of arterial thrombus stability and that ADAMTS13 helps to safeguard good vessel patency during a thrombotic event.

### C. Example 2: Recombinant ADAMTS13 Rescues Defective MCA Recanalization in ADAMTS13 KO Mice

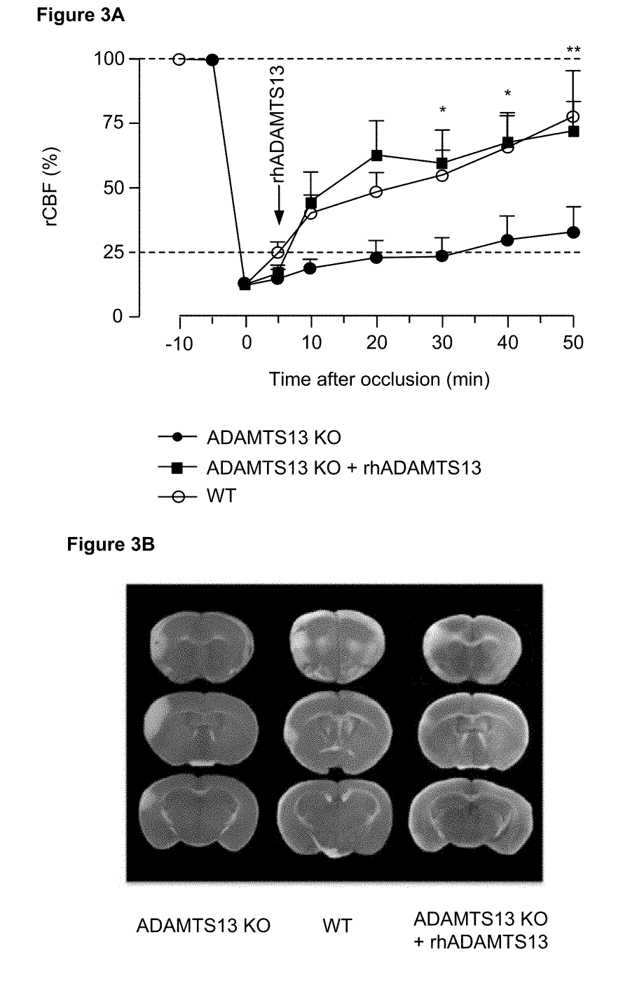

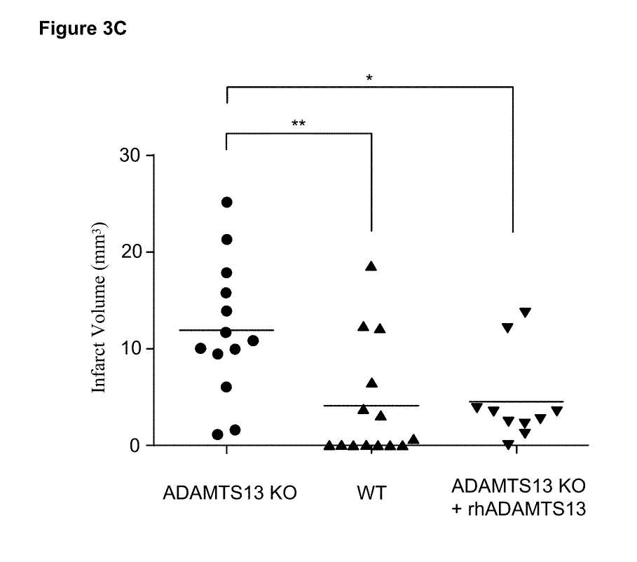

The above data suggest that ADAMTS13 can promote thrombus destabilization and enhance recanalization of occluded blood vessels. To further investigate this hypothesis, ADAMTS13 KO mice were treated with an intravenous injection of rhADAMTS13 (3500 U/kg) 5 minutes after threshold FeCl₃-induced thrombotic MCA occlusion. Post-occlusion pro-thrombolytic activity of rhADAMTS13 was followed by measuring rCBF via laser doppler flowmetry. Averaged blood flow was calculated at several time points after initial occlusion to quantify changes in rCBF over time (FIG. 3A). As expected, these rCBF profiles revealed a much better restoration of MCA blood flow in WT mice compared to ADAMTS13 KO mice, reaching statistical significance from 30 minutes onwards post-occlusion. At 50 minutes post-occlusion rCBF was restored to 78%±18% in WT mice opposed to only 33%±10% in the ADAMTS13 KO mice (p<0.01). Interestingly, however, when rADAMTS13 was administered to ADAMTS13 KO mice 5 minutes after occlusion, impaired recanalization could be rescued, resulting in efficient restoration of rCBF similar to WT mice (FIG. 3A). These data show that exogenous ADAMTS13 is able to destabilize an existing thrombus, thereby facilitating efficient thrombolysis and subsequent vessel recanalization.

### D. Example 3: Recombinant ADAMTS13-Mediated Restoration of MCA Blood Flow Protects ADAMTS13 KO Mice Against Ischemic Brain Injury

Next, studies were carried out to determine whether the observed differences in blood flow restoration had a physiological effect on ischemic brain injury. Therefore, mouse brains were isolated 24 hours post-occlusion and sections were stained with TTC to visualize cerebral infarctions (FIGS. 3B and 3C). As expected, infarctions were relatively small or even absent in WT animals (4.1 mm³±1.6 mm³). In line with poor MCA recanalization of ADAMTS13 KO mice, cerebral infarctions in these animals were significantly larger (11.9 mm³±1.9 mm³). Notably, in ADAMTS13 KO mice that received rhADAMTS13, infarct volumes were significantly reduced to similar values of WT animals (4.5 mm³±1.4 mm³). Hence, restoration of MCA blood flow by administration of rhADAMTS13 saves the brain from developing larger cerebral infarctions.

### E. Example 4: Recombinant ADAMTS13 Destabilizes Permanent Thrombotic Occlusions in WT Mice

The threshold injury used in the experiments described above allowed a more detailed dissection of ADAMTS13-related differences between ADAMTS13 KO and WT mice. However, initial thrombus formation in our thrombotic stroke model is influenced by the presence or absence of ADAMTS13, which could affect subsequent thrombus destabilization. To test the pro-thrombolytic effect of rhADAMTS13 in a more physiological setting, a permanent thrombotic occlusion was induced in the MCA of WT C57/B16J mice. To achieve this, the degree of injury was adjusted, using a larger filter paper saturated with 20% FeCl₃. As a result, the damaged area of the MCA was significantly larger, leading to permanent thrombotic occlusion of the mouse MCA (FIG. 1). In this model, no spontaneous recanalizations were observed for at least 2 hours after MCA occlusion.

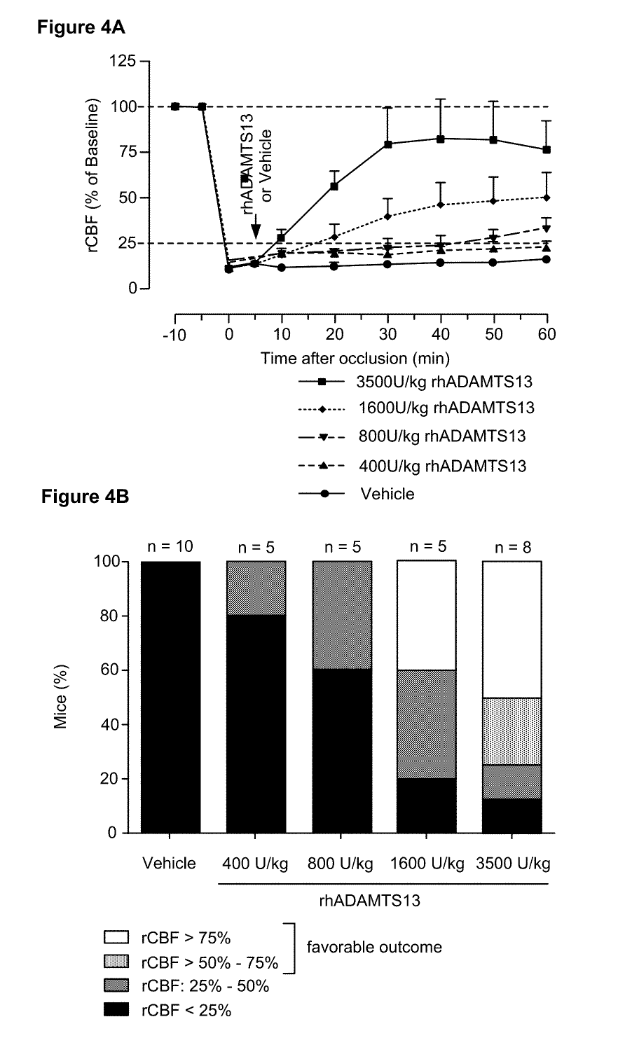

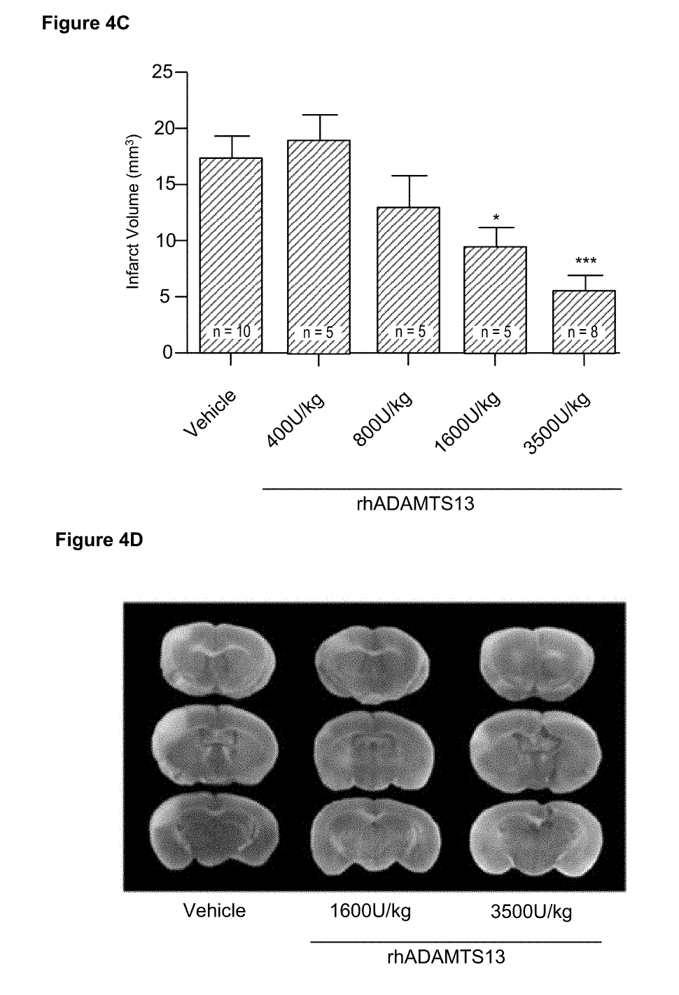

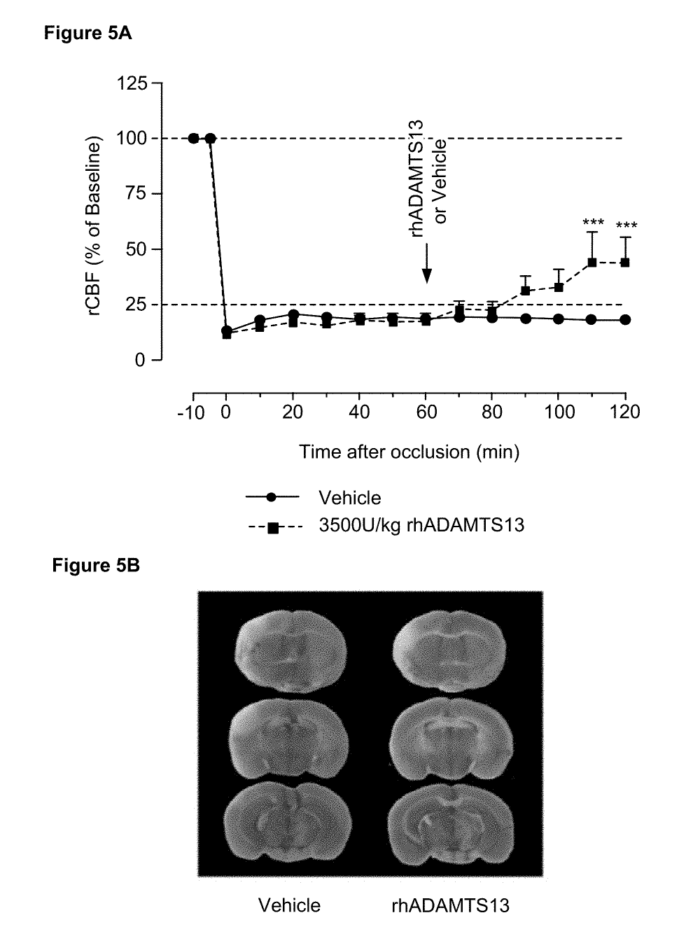

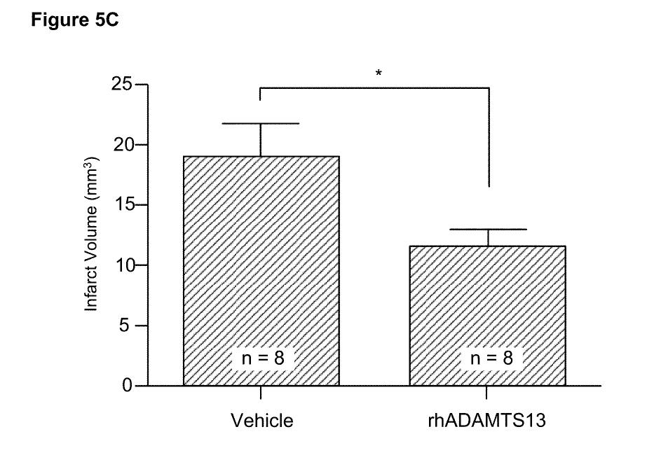

Using this model, a test was first carried out to determine whether rhADAMTS13 (3500 U/kg) could ameliorate MCA blood flow when administered 5 minutes after the start of occlusion. Strikingly, this dose of rhADAMTS13 was able to restore rCBF back to more than 75% within 25 minutes after injection (76.6%±15.9% of baseline values 60 minutes after occlusion, FIG. 4A). Vehicle administration had no effect on rCBF (16.9%±2.3% of baseline values 60 minutes after occlusion). Next lower doses of rhADAMTS13 were used to determine the minimally effective dose in this model. As shown in FIG. 5A, a dose-dependent effect was observed: 1600 U/kg still significantly improved rCBF when administered 5 minutes after occlusion (50.5%±13.6% of baseline values 60 min after occlusion) whereas a dose of 800 U/kg only showed a limited improvement in blood flow (33%±6% of baseline values 60 minutes after occlusion). A dose of 400 U/kg rhADAMTS13 was ineffective, as rCBF was not restored above 25% of baseline 60 minutes post-occlusion in the majority of mice (23%±3.6% of baseline values 60 minutes after occlusion). At the end of rCBF monitoring the grade of reperfusion was determined for each individual mouse (FIG. 4B). The lower doses of rhADAMTS13 (400 U/kg & 800 U/kg) only induced partial reperfusion (rCBF: 25%-50%) in 1 out of 5 mice and in 2 out of 5 mice respectively. It were only the higher doses of 1600 U/kg and 3500 U/kg of rhADMATS13 that were able to recover rCBF above 50% in 2 out of 5 mice and 6 out of 8 mice respectively.

Importantly, in line with the dose-dependent effect of blood flow restoration by ADAMTS13, a similar dose-response was seen on ischemic brain injury 24 hours post-occlusion (FIGS. 4C and 4D). Indeed, whereas administration of 400 U/kg rhADAMTS had no effect on infarct size compared to vehicle treatment (18.8 mm³±2.3 mm³ versus 17.3 mm³±2.2 mm³ respectively), administration of higher doses reduced cerebral infarct volumes. This protective effect was statistically significant for the two highest doses (1600 U/kg and 3500 U/kg) with infarct volumes of 9.4 mm³±1.6 mm³ and 5.3 mm³±1.7 mm³ respectively.

### F. Example 5: Delayed Administration of rhADAMTS13 Still Exerts a Prothrombolytic Effect Improving Stroke Outcome

To assess whether the thrombolytic potential of ADAMTS13 is also effective in a more clinically realistic broader time window, rhADAMTS13 (3500 U/kg) was intravenously injected 1 hour after stable occlusion of the MCA. Even after this prolonged period of thrombotic occlusion, rhADAMTS13 was still able to destabilize the thrombus, thereby partly restoring MCA patency (FIG. 5A). Although this effect was less stronger than early rhADAMTS13 administration, rCBF was still restored to 43.9%±11.7% of baseline values 60 min after rhADAMTS13 injection. Again, rCBF in the vehicle treated group remained at 18.2%±1.7% 60 min after injection. This partial restoration of blood flow was still sufficient to partly rescue the brain from the ischemic insult. Infarct sizes of mice treated with rhADAMTS13 1 hour post-occlusion were indeed significantly reduced when compared to mice that received vehicle (11.3 mm³±1.6 mm³ versus 18.8 mm³±2.9 mm³ respectively).

1. A method for recanalization of an occluded blood vessel in a subject having a cerebral infarction, comprising the step of administering to the subject a pharmaceutical composition comprising a therapeutically effective amount of isolated ADAMTS13 protein, thereby recanalizing the occluded blood vessel.

2. The method of claim 1, wherein the pharmaceutical composition is administered to the subject at a dose of about 40, 50, 60, 70, 80, 90, 100, 150, 200, 250, 300, 350, 400, 450, 500, 550, 600, 650, 700, 750, 800, 850, 900, 950, 1,000, 1,250, 1,500, 1,750, or 2,000 U/kg and/or
wherein the pharmaceutical composition is administered to the subject within 15, 30, 60, 90, 120, 180, 210, 240, 270 or 300 minutes of detection of the infarction.

3. A method for treating a cerebral infarction in a subject by recanalization of an occluded blood vessel in the subject, the method comprising the step of administering to the subject a pharmaceutical composition comprising a therapeutically effective amount of isolated ADAMTS13 protein, thereby treating the cerebral infarction.

4. The method of claim 3, wherein the pharmaceutical composition is administered to the subject at a dose of about 40, 50, 60, 70, 80, 90, 100, 150, 200, 250, 300, 350, 400, 450, 500, 550, 600, 650, 700, 750, 800, 850, 900, 950, 1,000, 1,250, 1,500, 1,750, or 2,000 U/kg and/or
wherein the pharmaceutical composition is administered to the subject within 15, 30, 60, 90, 120, 180, 210, 240, 270 or 300 minutes detection of the infarction.

5. The method of claim 1, wherein the pharmaceutical composition is administered to the subject at a dose of about 40, 50, 60, 70, 80, 90, 100, 150, 200, 250, 300, 350, 400, 450, 500, 550, 600, 650, 700, 750, 800, 850, 900, 950, 1,000, 1,250, 1,500, 1,750, or 2,000 U/kg; and
wherein the pharmaceutical composition is administered to the subject within 15, 30, 60, 90, 120, 180, 210, 240, 270 or 300 minutes of detection of the infarction.

6. A method for recanalization of an occluded blood vessel in a subject having a cerebral infarction, comprising the step of administering to the subject a pharmaceutical composition comprising a therapeutically effective amount of isolated ADAMTS13 protein, thereby recanalizing the occluded blood vessel,
wherein the pharmaceutical composition is administered to the subject at an amount that increases the level of the ADAMTS13 protein in the subject 1.1, 1.2, 1.3, 1.4, 1.5, 1.6, 1.7, 1.8, 1.9, 2, 3, 4, 5, 6, 7, 8, 9, 10, 15, or 20-fold greater than the level of ADAMTS13 protein in the subject prior to the administering.

7. The method of claim 6, wherein the pharmaceutical composition administered to the subject within 15, 30, 60, 90, 120, 180, 210, 240, 270 or 300 minutes of detection of the infarction.

8. The method of claim 1, wherein the regional cerebral blood flow in the subject is improved by at least 25% as compared to a control subject not having a cerebral infarction.

9. The method of claim 1, wherein the regional cerebral blood flow is improved by at least 50% as compared to the regional cerebral blood flow in the control subject.

10. The method of claim 1, wherein the regional cerebral blood flow is improved by at least 75% as compared to the regional cerebral blood flow in the control subject.

11. The method of claim 1, wherein the isolated ADAMTS13 protein is glycosylated.

12. The method of claim 1, wherein the isolated ADAMTS13 protein has a plasma half-life of more than 1 hour.

13. The method of claim 1, wherein the isolated ADAMTS13 protein is recombinantly produced by HEK293 cells.

14. The method of claim 1, wherein the isolated ADAMTS13 protein is recombinantly produced by CHO cells.

15. The method of claim 1, wherein the pharmaceutical composition is administered multiple times or by continuous infusion.

16. The method of claim 1, wherein said administration does not increase the level of hemorrhage, as compared to the level of hemorrhage in a subject not receiving the pharmaceutical composition.

17. The method of claim 1, wherein said administration reduces infarct volume.

18. The method of claim 17, wherein the infract volume is reduced by at least 50% compared to the infract volume in a control subject not having a cerebral infarction.

19. A method of improving the recovery of sensorimotor function in a subject that has experienced a cerebral infarction comprising the step of administering to the subject a pharmaceutical composition comprising a therapeutically effective amount of isolated ADAMTS13 protein, thereby improving the recovery of sensorimotor function,
wherein the regional cerebral blood flow in the subject is improved by at least 25% as compared to the regional cerebral blood flow in a control subject not having a cerebral infarction.
